(19) United States
(12) Patent Application Publication
(10) Pub. No.: US 2010/0240932 A1
(43) Pub. Date: Sep. 23, 2010

(54) Process for preparing dimethyl ether

(76) Inventors: Sam Ryong Park, Daejoen (KR); Gyung Rok Kim, Daejoen (KR); Gyoo Tae Kim, Daejoen (KR); Seung Hoon Oh, Daejoen (KR); Cheol Joong Kim, Daejoen (KR); Hyun Chul Choi, Daejoen (KR); Kyung Seok Noh, Daejoen (KR)

Correspondence Address: ABELMAN, FRAYNE & SCHWAB, 666 THIRD AVENUE, 10TH FLOOR, NEW YORK, NY, 10017, US

(21) Appl. No.: 12/310,529

(22) PCT Filed: Aug. 30, 2007
(86) PCT No.: PCT/KR2007/004181

§ 371 Date: Feb. 25, 2009

(30) Foreign Application Priority Data
Aug. 31, 2006 (KR) 10-2006-0083582

(51) Int. Cl. C07C 41/09

(52) U.S. Cl. 568/698

(57) ABSTRACT

Disclosed is a process for preparing dimethyl ether, including a) reacting methanol containing 0-80 mol % of water in the presence of a dehydration catalyst, b) transferring the reaction product into a single separation column, thus separating dimethyl ether, water, and unreacted methanol, c) withdrawing dimethyl ether and withdrawing unreacted methanol from the sidestream of the single separation column, and d) recyling the unreacted methanol to the a) reacting the methanol. Dimethyl ether may be prepared from water-containing methanol, and the separation and withdrawal of dimethyl ether, water, and unreacted methanol may be realized using a single column, thus reducing the investment cost and the operating cost.

### TECHNICAL FIELD

The present invention relates to a process for preparing dimethyl ether, and more particularly, to a process for economically preparing highly pure dimethyl ether, which is capable of decreasing the investment cost and the operating cost through a simple process, in which dimethyl ether (DME, purity: 99%) is prepared while maintaining a high conversion of dehydration of anhydrous methanol or hydrous methanol, and dimethyl ether, water, and unreacted methanol may be simultaneously separated from the reaction product using a single separation column.

### BACKGROUND ART

Dimethyl ether has high applicability as a principal material in the chemical industry, including as an aerosol spray propellant, and the useful value thereof as a fresh fuel is also high. Presently, dimethyl ether has the likelihood of serving as a fresh fuel for internal combustion engines, and it is thus required to develop a more economic process for the preparation thereof. The industrial preparation method of dimethyl ether includes the dehydration of methanol, as represented by Reaction 1 below:

2CH₃OH→CH₃OCH₃+H₂O Reaction 1

The reaction for preparing dimethyl ether through the dehydration of methanol is conducted at 200˜450° C., and mainly uses a solid acid catalyst. Typically, the preparation of dimethyl ether is performed by dehydrating anhydrous methanol (purity>98%) in the presence of a dehydration catalyst using a fixed-bed reactor, and then distilling the product.

The solid acid catalyst used in the preparation of dimethyl ether includes, for example, γ-alumina (Japanese Unexamined Patent Publication No. 1984-16845), silica-alumina (Japanese Unexamined Patent Publication No. 1984-42333), etc. However, because γ-alumina or silica-alumina is hydrophilic, water may be easily adsorbed on the surface thereof, thereby decreasing the number of activation sites of the catalyst, resulting in deteriorated catalytic activity. Therefore, the activity of the solid acid catalyst is significantly decreased when water is contained in methanol, which is used as a feedstock for the preparation of dimethyl ether. For this reason, methanol in which the water content is typically decreased to a level below hundreds of ppm has been used to prepare dimethyl ether.

However, methanol, which is prepared in the form of synthetic gas, contains 10-20% water as a by-product, and thus it is essentially required to remove water through distillation. Further, in unreacted methanol, which is withdrawn and reused in the course of the preparation of dimethyl ether, water produced through dehydration is contained in a relatively great amount, and this water must also be removed through distillation. Moreover, an Meₓ-H₍₁₋ₓ₎-zeolite catalyst (U.S. Pat. No. 6,740,783), known as a water-resistant catalyst, is used such that a strong hydrogen (H) acid site is replaced with a basic metal to thus eliminate such a strong acid site, thereby selectively preparing dimethyl ether. However, because the strength of the acid site depends strongly on the amount of metal that is replaced, it is difficult to reproduce the catalyst, and furthermore, the reaction zone within which it is possible to obtain dimethyl ether at a high yield is not wide.

Because the reaction for converting methanol into dimethyl ether progresses by means of an acid catalyst and the production of dimethyl ether corresponds to the formation of an intermediate in the course of the production of hydrocarbon, the activity and selectivity of the acid catalyst may vary depending on the acid site strength of the acid catalyst. For example, in the presence of a catalyst which carries a strong acid site, methanol is converted into dimethyl ether and an additional reaction is then conducted to produce hydrocarbon as a by-product, whereas, in the presence of a catalyst which carries only a weak acid site, the conversion of methanol into dimethyl ether is insufficient, attributable to the low activity of the catalyst.

As illustrated in FIG. 1, the method of producing dimethyl ether on an industrial scale typically includes dehydrating methanol in the presence of a dehydration catalyst using a fixed-bed reactor, separating dimethyl ether from the reaction product through a first distillation column, and separating water and unreacted methanol through a second distillation column. The process for preparing dimethyl ether using two distillation columns is disclosed in U.S. Pat. Nos. 4,560,807, 5,750,799, and 6,924,399.

U.S. Pat. No. 5,750,799 and Korean Unexamined Patent Publication No. 1998-702932 disclose a method of recycling methanol, containing water, for use. However, when methanol is produced, hydrous methanol containing a great amount of water is produced before the purification, and thus is unsuitable for use as a feedstock.

In regard to the purification of dimethyl ether, U.S. Pat. Nos. 5,027,511 and 4,802,956 disclose a method of obtaining highly pure and odorless dimethyl ether, by additionally providing a sidestream withdrawal tray for removing impurities between a feed-in tray and a dimethyl ether separation tray in a first dimethyl ether separation column for separating dimethyl ether. Further, a recycling distillation column for separating water/methanol is provided after two dimethyl ether separation columns. Consequently, examples for the preparation of dimethyl ether using a single separation column have not yet been reported.

### DISCLOSURE

### Technical Problem

Leading to the present invention, intensive and thorough research carried out by the present inventors, aiming to solve the problems encountered in the related art, resulted in the finding that dimethyl ether, water and unreacted methanol may be simultaneously separated through a single separation column using a water-resistant catalyst having catalytic activity that is not decreased even when a feedstock containing water is used, thus preparing dimethyl ether at a high yield while generating economic benefits in terms of cost.

Accordingly, the present invention provides a process for economically preparing dimethyl ether.

### Technical Solution

According to the present invention, there is a process for preparing dimethyl ether, including a) reacting methanol containing 0˜80 mol % of water in the presence of a dehydration catalyst in which some or all of sodium cations (Na⁺) of Na-type zeolite are substituted with phosphorus (P), as represented by Formula 1 below:

NaₓP₍₁₋ₓ₎Z Formula 1

wherein Na is a sodium cation, P is a phosphorus cation, x is a sodium cation content ranging from 0 to 99 mol %, and Z is hydrophobic zeolite, in which the ratio of SiO₂/Al₂O₃ ranges from 5 to 200; b) transferring the reaction product into a single separation column, thus separating dimethyl ether, water, and unreacted methanol; and c) withdrawing dimethyl ether and withdrawing unreacted methanol from the sidestream of the single separation column.

### Advantageous Effects

According to the present invention, highly pure dimethyl ether (purity>99%) may be prepared from methanol containing a maximum of 80 mol % of water, unlike conventional techniques, and a high conversion (>75%) of methanol dehydration may be maintained.

Further, unreacted methanol may be recycled without the need for a process for removing water, thus efficiently coping with a change in water content of the feedstock, and a single separation column may be used, instead of two separation columns, which are conventionally used, thus decreasing the size of the apparatus. Furthermore, the consumption of steam and cooling water may be decreased by 10% or more, compared to conventional processes, thereby reducing the investment cost and the operating cost, resulting in high industrial availability.

### DESCRIPTION OF DRAWINGS

FIG. 1 illustrates a process for preparing dimethyl ether, according to a conventional technique;

FIG. 2 illustrates a process for preparing dimethyl ether, according to a first embodiment of the present invention;

FIG. 3 illustrates a process for preparing dimethyl ether, according to a second embodiment of the present invention; and

FIG. 4 illustrates a process for preparing dimethyl ether, according to a third embodiment of the present invention.

### DESCRIPTION OF THE REFERENCE NUMERALS IN THE DRAWINGS

11: evaporator

12: reactor

13: dimethyl ether separation column

14: methanol separation column

15: dimethyl ether, water and methanol separation column

16: flash drum

DME: dimethyl ether

MeOH: unreacted methanol

### Best Mode

Hereinafter, a detailed description will be given of the present invention.

As mentioned above, the present invention provides a process for economically preparing dimethyl ether using a single column provided with a recycling sidestream to reduce the investment cost and the operating cost.

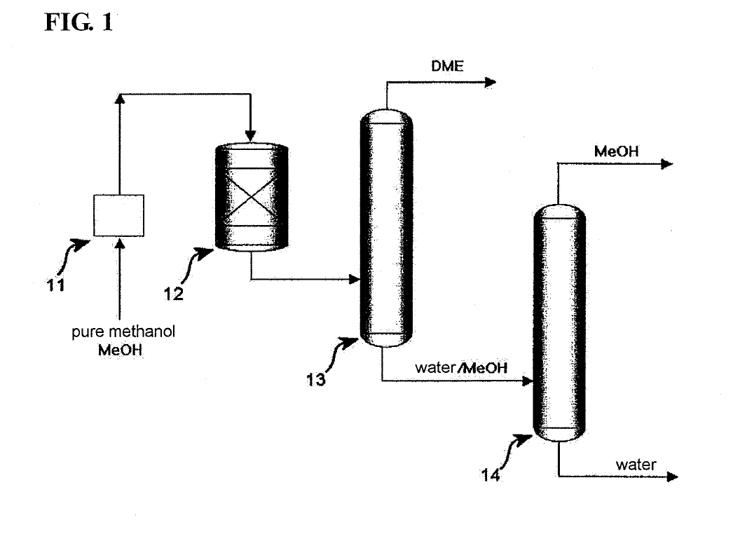

A description of the process for preparing dimethyl ether according to the conventional technique follows, with reference to FIG. 1. Conventionally, as the water content in a feedstock is increased, the activity of the catalyst loaded into a reactor 12 is decreased, undesirably lowering the yield. Ultimately, anhydrous methanol (purity>98%) should be used as a feedstock. In the reactor 12, filled with the catalyst, anhydrous methanol is reacted, after which the reaction product is transferred into a first separation column 13 for withdrawing dimethyl ether, thus withdrawing dimethyl ether. Meanwhile, the residual reaction product is transferred into a second separation column 14, thus withdrawing water and unreacted methanol. In this way, a two-column system is conventionally applied, and the yield is maintained at a level of 70% or less. The second separation column is typically a multi-tray column composed of 50 or more trays, in order to decrease the water content of the unreacted methanol that is recycled.

However, according to the present invention, not only anhydrous methanol but also hydrous methanol may be used as a feedstock for dimethyl ether production. More specifically, in the present invention, methanol, in which water is contained in an amount of 0˜80 mol %, and preferably 10˜30 mol %, may be used as a feedstock.

The process for preparing dimethyl ether according to the present invention includes reacting hydrous methanol containing 0˜80 mol % of water in the presence of a dehydration catalyst, transferring the reaction product into a single separation column to thus separate dimethyl ether, water, and unreacted methanol, withdrawing dimethyl ether and withdrawing water and methanol from a sidestream, and recycling the unreacted methanol.

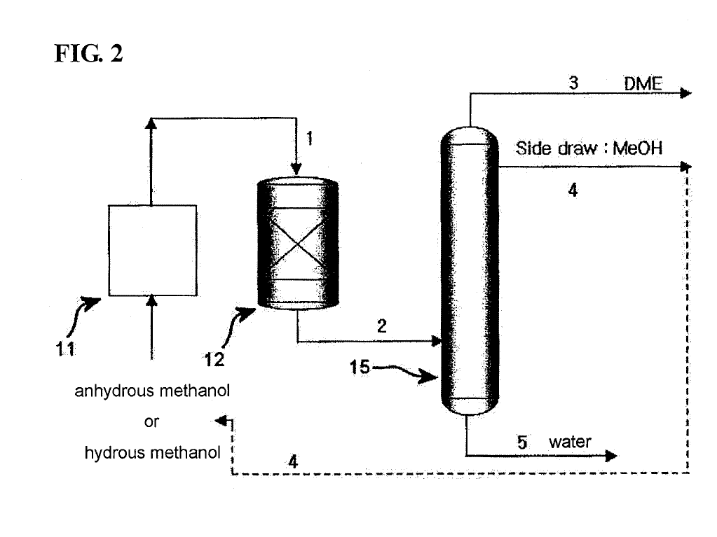

According to a first embodiment of the present invention, as illustrated in FIG. 2, anhydrous or hydrous methanol, serving as a feedstock, is supplied into a reactor 12 filled with a dehydration catalyst to thus be reacted, after which the reaction product is transferred into a separation column 15 along a stream 2. As such, dimethyl ether is withdrawn along a stream 3 from the top of the column, and unconverted methanol, containing a small amount of dimethyl ether, is recycled along a stream 4 from the upper sidestream of the column.

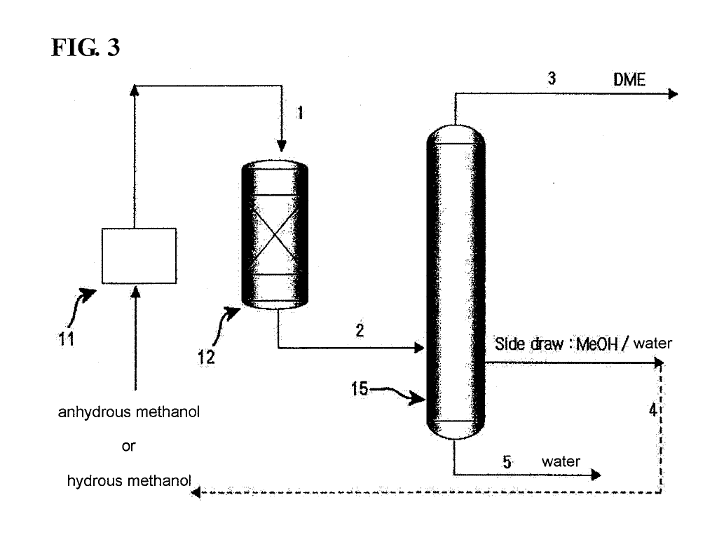

According to a second embodiment of the present invention, as illustrated in FIG. 3, dimethyl ether is withdrawn along a stream 3 from the top of a separation column 15, while unreacted methanol containing water may be recycled along a stream 4 from the lower sidestream of the column.

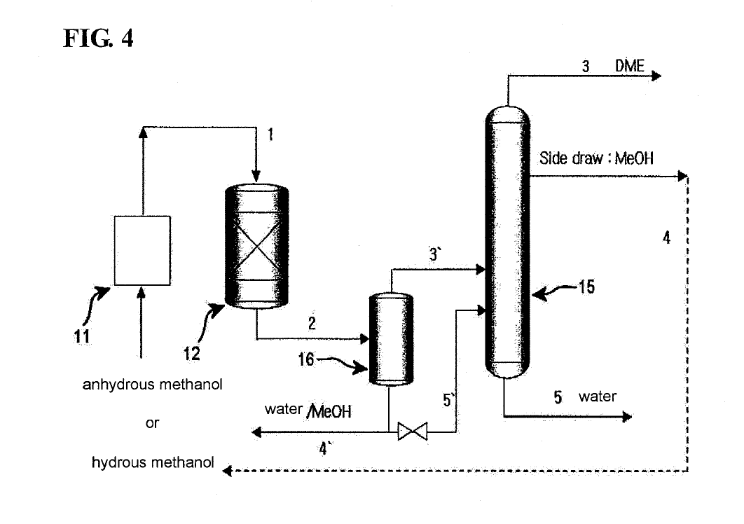

According to a third embodiment of the present invention, as illustrated in FIG. 4, a flash drum 16, which incurs only a very small investment cost, may be provided after a reactor 12, in order to decrease the volume of the separation column 15. A gas phase, composed of dimethyl ether, unreacted methanol and water, is transferred to the separation column 15 along a stream 3′ from the top of the flash drum 16, whereas a liquid phase, in which water and unreacted methanol, containing a small amount of dimethyl ether, are mainly present, is recycled along a stream 4′ from the bottom thereof. Further, dimethyl ether is withdrawn along a stream 3 from the separation column 15, and the unreacted methanol is recycled along a stream 4.

The effluent discharged from the reactor includes water, methanol, and dimethyl ether mixed together, and is present in a gaseous state (e.g., even if the reaction effluent at about 290° C. is subjected to heat exchange with a reactor inlet flow, it is in a gaseous state at about 170° C. when supplied into the separation column). Thus, when all the effluent is supplied into the separation column, the size of the column and the heat duties are increased, undesirably increasing the apparatus investment cost and the operating cost. For this reason, the flash drum is used. In this way, when the reaction mixture is partially condensed using the flash drum (functioning to decrease the temperature, that is, to realize partial condensation) before it is supplied into the column, part of the condensate is recycled and the residual condensate and the flash overhead gas are supplied into the separation column and separated therethrough. Due to the difference in properties, including the boiling point, between dimethyl ether and water/methanol, the flash condensate is composed mainly of water or water/methanol, and includes less than 1% dimethyl (90% or more of the condensate is water, and the balance thereof is composed mainly of methanol).

In the present invention, the dehydration catalyst is a catalyst in which some or all of the sodium cations of Na-type zeolite are substituted with phosphorus (P), as represented by Formula 1 below:

NaₓP₍₁₋ₓ₎Z Formula 1

wherein Na is a sodium cation, P is a phosphorus cation, x is a sodium cation content ranging from 0 to 99 mol %, and Z is hydrophobic zeolite, in which the ratio of SiO₂/Al₂O₃ ranges from 5 to 200, and preferably from 10 to 100.

The catalyst represented by Formula 1 may maintain high catalytic activity for a long period of time without inactivation by water, and thus is responsible for effectively dehydrating a methanol feedstock. Further, the strength of the acid site may be controlled by substituting some or all of the sodium cations (Na⁺) of Na-type zeolite with the phosphorus (P) ion, thereby increasing the activity of the catalyst, resulting in greatly improved selectivity of dimethyl ether.

In the catalyst of the present invention, zeolite Z is hydrophobic zeolite based on USY, mordenite, ZSM, Beta, etc., in which the ratio of SiO₂/Al₂O₃ ranges from 5 to 200. When the ratio of SiO₂/Al₂O₃ exceeds 200, the number of acid sites is too low, or almost zero, such that methanol cannot be effectively dehydrated. On the other hand, when the ratio is less than 5, the acid strength of the acid site is high, and thus the operable temperature range becomes narrow.

The catalyst according to the present invention is a catalyst able to maintain the same activity and reaction zone with respect to anhydrous methanol/hydrous methanol (containing 0˜80 mol % of water). Hence, in the presence of such a catalyst, even when the recycled methanol stream is supplied in a state of containing water in the reactor, the same activity and reaction zone may be maintained, thereby making it possible to use the single separation column without the need for complete separation of water/methanol.

Below, the process for preparing the dimethyl ether according to the present invention is described in more detail. The dehydration catalyst is loaded into the reactor, and is then pre-treated at 200˜500° C. while inert gas such as nitrogen is supplied at a flow rate of 20˜100 ml/g-catalyst/min, before methanol dehydration. In the presence of the pre-treated catalyst, anhydrous methanol or hydrous methanol, specifically methanol, in which water is contained in an amount of 0˜80 mol %, and preferably 10˜30 mol %, flows into the reactor.

The reaction temperature is maintained at 150˜500° C. When the reaction temperature is lower than 150° C., the conversion is decreased because the reaction rate is too slow. On the other hand, when the reaction temperature exceeds 500° C., ethylene is produced through continuous dehydration of the produced dimethyl ether, undesirably drastically increasing the temperature of the catalyst bed. The reaction pressure is maintained at 1˜100 atm. When the pressure exceeds 100 atm, problems related to the operation of the reactor are undesirably caused. Further, methanol dehydration may be conducted at an LHSV (Liquid Hourly Space Velocity) of 0.05˜50 h⁻¹ based on pure methanol. When the LHSV is less than 0.05 h⁻¹, the reaction productivity becomes too low. On the other hand, when the LHSV exceeds 50 h⁻¹, the feedstock is brought into contact with the catalyst for too short a time, undesirably decreasing the conversion. The reactor includes gas fixed-bed reactors, fluidized-bed reactors, liquid slurry reactors and the like. Regardless of what kind of reactor is used, the same effect may be attained.

Next, the reaction product, produced in the reactor, is transferred into the separation column, such that dimethyl ether is separated and withdrawn from the top of the column, and water is withdrawn from the bottom stream of the column. Further, unreacted methanol or a mixture of water/methanol is withdrawn from the sidestream thereof to thus be recycled as the feedstock. More specifically, dimethyl ether is withdrawn from the top of the single separation column and water is withdrawn from the bottom thereof; and furthermore, methanol or a mixture of water/methanol is withdrawn from between the feed-in tray and the overhead tray, among all of the trays of the column. As such, as the sidestream tray is provided at an upward position in the column, the purity of the methanol is increased. For example, in the case of the feed-in to tray 36 among the total of 56 trays, recycled methanol having a purity of 98% or more may be withdrawn from tray 9. When the level of the sidestream tray is below tray 9, that is, when the sidestream is provided close to the feed-in tray, methanol containing a greater amount of water is withdrawn.

According to the present invention, even though methanol containing water is supplied as a feedstock, the conversion may be maintained at a level greater than 75% based on the amount of methanol, and dimethyl ether having a purity of 99% or more (in particular, for fuels) may be prepared at a high yield. Further, the process of the present invention is advantageous because water and methanol may be separately recycled through a single separation column, and therefore, economic benefits able to reduce the investment cost and the operating cost are generated.

### Mode for Invention

A better understanding of the present invention may be obtained through the following examples, which are set forth to illustrate, but are not to be construed as the limit of the present invention.

### Example 1

300 liters of an NaPZSM-5 catalyst was loaded into a fixed-bed reactor. In this state, the catalyst was pre-treated at 400° C. for 1 hour while nitrogen was supplied at a flow rate of 18.5 Nm³/min, after which the temperature of the reactor was set to 250° C. Subsequently, hydrous methanol containing 20 mol % of water was supplied as a feedstock under conditions of reaction pressure of 10 atm and reaction temperature of 250° C., and was combined with a recycling stream and thus passed through the catalyst bed at LHSV of 5˜10 h⁻¹.

The reaction product (DME, water, unreacted MeOH) discharged from the reactor was transferred into a single separation column. The operation conditions of the single separation column were set as follows, that is, the number of total trays was 56, the pressure of the top tray was about 10 kg/cm², the temperature at the bottom of the column was about 184° C., and the temperature at the top of the column was about 48° C. After the feed-in to tray 36, the dimethyl ether product from the overhead stream, water from the bottom of the column, and recycled methanol (purity: 98% or more) from tray 9 were withdrawn. The purity of the discharged recycled methanol was 98% or more.

The flow rates and the amounts of methanol, dimethyl ether and water of respective streams in FIG. 2 are shown in Table 1 below. As such, single-pass conversion of methanol passing through the reactor was about 80%, and total conversion and yield, including recycling, were about 99% or more (side-reaction product: less than 1%, that is, selectivity: 99% or more).

TABLE 1

| | Stream No. | | | | | |
|---|---|---|---|---|---|---|
| | Feed | 1 | 2 | 3 | 4 | 5 |
| Flow Rate (kg/hr) | 1,000 | 1,225.6 | 1,225.6 | 630.3 | 225.5 | 369.7 |
| Methanol (mol %) | 80.0 | 83.0 | 16.6 | 0.0 | 98.0 | 0.0 |
| DME (mol %) | 0.0 | 0.3 | 33.6 | 100.0 | 2.0 | 0.0 |
| Water (mol %) | 20.0 | 16.7 | 49.8 | 0.0 | 0 | 100.0 |

### Example 2

300 liters of an NaPZSM-5 catalyst was loaded into a fixed bed reactor. In this state, the catalyst was pre-treated at 400° C. for 1 hour while nitrogen was supplied at a flow rate of 18.5 Nm³/min, after which the temperature of the reactor was set to 250° C. Subsequently, hydrous methanol containing 20 mol % of water was supplied as a feedstock under conditions of reaction pressure of 10 atm and reaction temperature of 250° C., and was combined with a recycling stream, and thus passed through the catalyst bed at LHSV of 5˜10 h⁻¹.

The reaction product (DME, water, unreacted MeOH), discharged from the reactor, was transferred into a single separation column. The operation conditions of the single separation column were set as follows, that is, the number of total frays was 56, the pressure of the top tray was about 10 kg/cm², the temperature at the bottom of the column was about 184° C., and the temperature at the top of the column was about 48° C. After the feed-in to fray 34, the dimethyl ether product from the overhead stream, water from the bottom of the column, and a mixture of water/methanol (methanol: about 91%, water: 7%) from tray 43 were withdrawn, and the methanol containing water was recycled to a feed surge drum.

The flow rates and the amounts of methanol, dimethyl ether and water of respective streams in FIG. 3 are shown in Table 2 below. As such, the single-pass conversion of methanol passing through the reactor was about 80%, and the total conversion and yield, including recycling, were about 99% or more (side-reaction product: less than 1%, that is, selectivity: 99% or more).

TABLE 2

| | Stream No. | | | | | |
|---|---|---|---|---|---|---|
| | Feed | 1 | 2 | 3 | 4 | 5 |
| Flow Rate (kg/hr) | 1,000 | 1,236.8 | 1,236.8 | 628.7 | 236.8 | 371.3 |
| Methanol (mol %) | 80 | 82.1 | 16.4 | 0 | 91.7 | 0.3 |
| DME (mol %) | 0 | 0.3 | 33.4 | 100 | 1.7 | 0 |
| Water (mol %) | 20 | 17.6 | 50.2 | 0 | 6.6 | 99.7 |

### Example 3

300 liters of an NaPZSM-5 catalyst was loaded into a fixed-bed reactor. In this state, the catalyst was pre-treated at 400° C. for 1 hour while nitrogen was supplied at a flow rate of 18.5 Nm³/min, after which the temperature of the reactor was set to 250° C. Subsequently, hydrous methanol containing 20 mol % of water was supplied as a feedstock under conditions of reaction pressure of 10 atm and reaction temperature of 250° C., and was combined with a recycling stream and thus passed through the catalyst bed at LHSV of 5˜10 h⁻¹.

The reaction product (DME, water, unreacted MeOH, 10 kg/cm², near 300° C.), discharged from the reactor, was in a gaseous phase of about 170˜180° C. after heat exchange, and was fed into a flash drum before being transferred into a single separation column. Through the flash drum, the temperature was decreased to about 153° C., and partial condensation occurred. About 20% by the mass ratio of the flash drum feed was condensed to a liquid phase (water: about 93%, methanol: about 6.6%), and 50% thereof was recycled to the feed surge drum, and the residual condensate and the flash overhead gas were separately supplied into the single separation column. The single separation column was composed of a total of 56 trays, the pressure of the top tray was about 10 kg/cm², the temperature at the bottom of the column was about 184° C., and the temperature at the top of the column was about 48° C. The flash drum overhead gas was fed into tray 34, and the liquid condensate was fed into tray 36. As in Example 1, the dimethyl ether product from the overhead stream, water from the bottom of the column, and recycled methanol (purity: 98% or more) from tray 9 were withdrawn. The purity of the discharged recycled methanol was 98% or more.

The flow rates and the amounts of methanol, dimethyl ether and water of respective streams in FIG. 4 are shown in Table 3 below. As such, the single-pass conversion of methanol passing through the reactor was about 80%, and the total conversion and yield, including recycling, were about 99% or more (side-reaction product: less than 1%, that is, selectivity: 99% or more).

TABLE 3

| | Stream No. | | | | | | | | |
|---|---|---|---|---|---|---|---|---|---|
| | Feed | 1 | 2 | 3′ | 4′ | 5′ | 3 | 4 | 5 |
| Flow Rate (kg/hr) | 1,000 | 1,341 | 1,341 | 1,081 | 130.2 | 130.2 | 630.3 | 211.0 | 369.6 |
| Methanol (mol %) | 80 | 71.9 | 14.4 | 17.5 | 6.6 | 6.6 | 0 | 97.9 | 0 |
| DME (mol %) | 0 | 0.3 | 29.0 | 40.8 | 0.1 | 0.1 | 100 | 2.1 | 0 |
| Water (mol %) | 20 | 27.8 | 56.6 | 41.7 | 93.3 | 93.3 | 0 | 0 | 100 |

1. A process for preparing dimethyl ether, comprising:
a) reacting methanol containing 0˜80 mol % of water in presence of a dehydration catalyst in which some or all of sodium cations (Na⁺) of Na-type zeolite are substituted with phosphorus (P), as represented by Formula 1 below:
NaₓP₍₁₋ₓ₎Z Formula 1
wherein Na is a sodium cation; P is a phosphorus cation; x is a sodium cation content ranging from 0 to 99 mol %; and Z is hydrophobic zeolite, in which a ratio of SiO₂/Al₂O₃ ranges from 5 to 200;
b) transferring the reaction product into a single separation column, thus separating dimethyl ether, water, and unreacted methanol; and
c) withdrawing dimethyl ether and withdrawing unreacted methanol from a sidestream of the single separation column.

2. The process according to claim 1, wherein the withdrawing the unreacted methanol in c) is conducted by withdrawing the unreacted methanol along with a small amount of dimethyl ether from an upper sidestream of the single separation column.

3. The process according to claim 1, wherein the withdrawing the unreacted methanol in c) is conducted by withdrawing the unreacted methanol in a form of containing water from a lower sidestream of the single separation column.

4. The process device according to claim 1, wherein the b) transferring the reaction product further comprises passing the reaction product through a flash drum, before transferring the reaction product into the single separation column.

5. The process device according to claim 1, wherein the c) withdrawing the dimethyl ether and withdrawing the unreacted methanol further comprises recycling the unreacted methanol to the a) reacting the methanol.

6. The process device according to claim 1, wherein the a) reacting the methanol is conducted at a reaction temperature ranging from 150° C. to 500° C., a reaction pressure ranging from 1 atm to 100 atm, and a liquid hourly space velocity ranging from 0.05 h⁻¹ to 50 h⁻¹.
